(19) United States
(12) Patent Application Publication
(10) Pub. No.: US 2009/0263328 A1
(43) Pub. Date: Oct. 22, 2009

(54) FLUORESCENCE OBSERVATION APPARATUS AND FLUOROSCOPY METHOD

(75) Inventors: Chika NAKAJIMA, Tokyo (JP); Yoshihisa TANIKAWA, Tokyo (JP); Nobuyuki NAGASAWA, Gunma (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/424,069

(22) Filed: Apr. 15, 2009

(30) Foreign Application Priority Data
Apr. 17, 2008 (JP) 2008-108036

(51) Int. Cl. A61K 49/00; C12M 1/34; A61P 35/00

(52) U.S. Cl. 424/9.6; 435/288.7

(57) ABSTRACT

A site of interest to be observed is clearly observed in an image during fluoroscopy of a small laboratory animal, even when the fluorescence is extremely low. The invention provides a fluorescence observation apparatus including a light source that emits excitation light; an optical system that irradiates an image-acquisition site on a small laboratory animal with the excitation light from the light source; a light-blocking unit that blocks light in a prescribed region of the small laboratory animal or in an image of the prescribed region; an image-acquisition unit that acquires a fluorescence image of the small laboratory animal; and a control unit configured to identify a high-fluorescence region having a prescribed fluorescence level or above in the fluorescence image of the small laboratory animal acquired by the image-acquisition unit and to control the light-blocking unit so as to block light at the identified high-fluorescence region.

### BACKGROUND OF THE INVENTION

1. Field of the Invention

The present invention relates to a fluorescence observation apparatuses and a fluoroscopy method for in vivo observation.

This application is based on Japanese Patent Application No. 2008-108036, the content of which is incorporated herein by reference.

2. Description of Related Art

Known fluorescence observation apparatuses in the related art irradiate a small laboratory animal, such as a mouse, with excitation light and observe fluorescence produced by a lesion such as cancer tissue or the like (for example, see U.S. Pat. No. 5,650,135).

Fluoroscopy observes fluorescence with relatively high brightness compared with luminoscopy, and therefore has advantages such as more clear observed images and superior ease of observation.

One known method of performing fluoroscopy of lesions, such as cancer tissue, involves administering a small laboratory animal with a fluorescent contrast agent exhibiting high accumulation in tumor tissue or a fluorescent contrast agent having a long retention time in blood vessels (for example, see Japanese Unexamined Patent Application, Publication No. 2003-261464). A site of interest can be easily tagged with the fluorescent contrast agent by administering it into the body by injection, spraying, application, etc. intravascular (veins and arteries), orally, interperitoneally, subdermally, intradermally, intravesically, intrabronchially, and so forth. With fluoroscopy using a fluorescent contrast agent, it is extremely simple to detect lesions compared with diagnostic imaging with X-ray radiography, MRI, ultrasound imaging etc.

However, with the method involving tagging the site of interest with a fluorescent contrast agent by intravascular administration, which is a method that is often used to administer fluorescent contrast agents to small laboratory animals, followed by fluoroscopy, eventually the fluorescent contrast agent that is subjected to glomerular filtration in the kidney and is not reabsorbed temporarily accumulates in the bladder. Therefore, fluorescence may be detected from the bladder. In addition, because the fluorescent contrast agent also accumulates in the liver, fluorescence may also be detected from the liver.

In other words, due to the properties of the fluorescent contrast agent, there is a problem in that fluorescence may end up being detected from the bladder, liver, etc., which are not the site to be targeted for observation.

Depending on the elapsed time after administering the fluorescent contrast agent to the small laboratory animal, the amount accumulated in the bladder etc. may be large, and the fluorescence level may be stronger than the fluorescence detected from the tumor tissue or blood vessels which are the sites to be observed.

If a fluorescence image is acquired under such conditions, because the bladder etc., which is not the observation site, emits extremely strong fluorescence, by acquiring an image with an exposure time according to the fluorescence level of the tumor tissue or blood vessels, the portion corresponding to the bladder etc. in the acquired image may become saturated, and the fluorescence of microvessels in the vicinity of the bladder may become drowned out. Another problem is that, if the exposure time for image acquisition is set according to a portion showing strong fluorescence, such as the bladder, which is not the site of interest, it is impossible to clearly detect the extremely weak fluorescence from the site of interest.

The same problem also occurs not just in administering a fluorescent contrast agent, but also when food consumed by the small laboratory animal is present in the stomach or intestine, since the stomach or intestine may strongly emit light due to autofluorescence of that food. There is also a possibility of the same problem occurring due to autofluorescence or reflected light of a jig used for the purpose of restraining the small animal.

### BRIEF SUMMARY OF THE INVENTION

The present invention has been conceived in light of the circumstances described above, and an object thereof is to provide a fluorescence observation apparatus and a fluoroscopy method that can clearly observe a site of interest to be observed in an image obtained during fluoroscopy of a small laboratory animal, even when the fluorescence is extremely low.

In order to achieve the above objects, the present invention provides the following solutions.

A first aspect of the present invention is a fluorescence observation apparatus including a light source that emits excitation light; an optical system that irradiates an image-acquisition site on a small laboratory animal with the excitation light from the light source; a light-blocking unit that blocks light in a prescribed region of the small laboratory animal or in an image of the prescribed region; an image-acquisition unit that acquires a fluorescence image of the small laboratory animal; and a control unit configured to identify a high-fluorescence region having a prescribed fluorescence level or above in the fluorescence image of the small laboratory animal acquired by the image-acquisition unit and to control the light-blocking unit so as to block light at the identified high-fluorescence region.

The aspect described above may further comprise a display unit that displays the fluorescence image of the small laboratory animal acquired by the image-acquisition unit; and a specifying unit configured to specify the high-fluorescence region in the fluorescence image displayed by the display unit.

The light-blocking unit may be a liquid-crystal filter or a digital micromirror device disposed at a position substantially conjugate with respect to the image-acquisition site on the small laboratory animal.

The light-blocking unit may be a galvanometer mirror disposed at a position substantially conjugate with respect to a pupil position of the optical system.

A second aspect of the present invention is a fluoroscopy method including an irradiating step of irradiating an image-acquisition site on a small laboratory animal with excitation light; a first image-acquiring step of acquiring a fluorescence image of the small laboratory animal; an extracting step of extracting a high-fluorescence region with a prescribed fluorescence level or above in the fluorescence image acquired in the first image-acquiring step; a light-blocking step of blocking light at a high-fluorescence site in the small laboratory animal, corresponding to the high-fluorescence region extracted in the extracting step, or in an image of the high-fluorescence site; and a second image-acquiring step of acquiring a fluorescence image of the small laboratory animal when the light at the high-fluorescence site of the small laboratory animal, corresponding to the high-fluorescence region, or in the image of the high-fluorescence site is blocked in the light-blocking step.

The present invention affords an advantage in that it is possible to clearly observe a site of interest to be observed in an image during fluoroscopy of a small laboratory animal, even when the fluorescence is extremely low.

### BRIEF DESCRIPTION OF THE SEVERAL VIEWS OF THE DRAWINGS

FIG. 1 is a diagram showing, in outline, the configuration of a fluorescence observation apparatus according to a first embodiment of the present invention.

FIG. 2 is a diagram showing a fluorescence image displayed on a display unit of the fluorescence observation apparatus in FIG. 1.

FIG. 3 is a diagram showing a light blocking part in a light blocking unit in the fluorescence observation apparatus in FIG. 1.

FIG. 4 is a diagram showing, in outline, a fluorescence observation apparatus according to a second embodiment of the present invention.

FIG. 5 is a diagram showing, in outline, a fluorescence observation apparatus according to a third embodiment of the present invention.

FIG. 6 is a diagram showing, in outline, a fluorescence observation apparatus according to a fourth embodiment of the present invention.

FIG. 7 is a diagram showing, in outline, a fluorescence observation apparatus according to a fifth embodiment of the present invention.

### DETAILED DESCRIPTION OF THE INVENTION

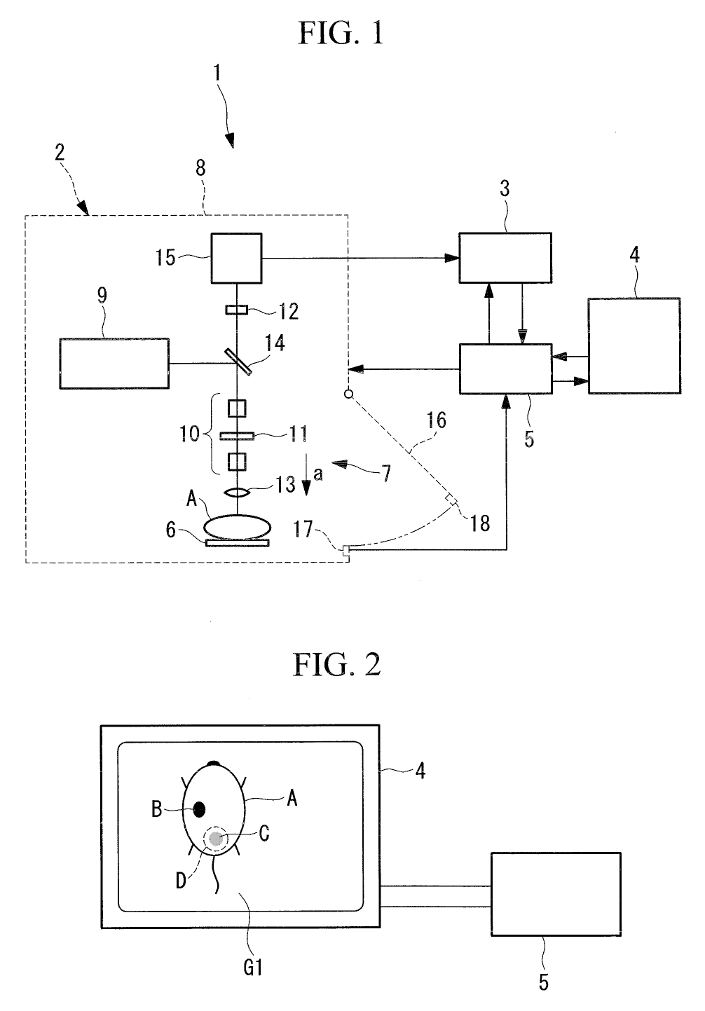

A fluorescence observation apparatus 1 according to a first embodiment of the present invention will be described below with reference to FIGS. 1 to 3.

As shown in FIG. 1, the fluorescence observation apparatus 1 according to this embodiment includes a fluorescence observation apparatus main body 2, an image storage unit 3, a display unit 4, and a controller 5 that controls them.

The fluorescence observation apparatus main body 2 includes a stage 6 for mounting a small laboratory animal, for example, a mouse A; an observation optical system 7; and a case 8 accommodating the observation optical system 7 to shield it from external light.

The observation optical system 7 includes an illumination device 9 that supplies illumination light; a dichroic mirror 14 that reflects the illumination light onto an optical axis a of the observation optical system 7; a relay optical system 10 that relays the illumination light; a light blocking unit 11 that is disposed at a substantially conjugate position with respect to an observation site on the mouse A and that blocks light at a specific region; an objective lens 13 that irradiates the mouse A on the stage 6 with the illumination light and that collects reflected light returning from the mouse A and fluorescence from the mouse A; an image-acquisition optical system 12 that images on an image-acquisition unit 15 the reflected light collected by the objective lens 13 and returning via the light blocking unit 11 and the relay optical system 10; and the image-acquisition unit 15 that acquires an image thereof to obtain a fluorescence image.

The illumination device 9 is equipped with a lamp serving as a light source for radiating illumination light (not shown), an excitation filter having characteristics that allow transmission of only specific wavelengths, and a shutter for blocking the illumination light. A plurality of the excitation filters are provided, and any one of them can be disposed on the optical axis.

The image-acquisition unit 15 is provided with an absorption filter having characteristics that allow transmission of only specific wavelengths (not shown).

An openable/closable door 16 is provided in the case 8, in the vicinity of the stage 6. The door 16 is provided with a sensor 17 that detects when the door 16 is closed. Reference numeral 18 is a detection piece to be detected by the sensor 17.

The image storage unit 3 can store a fluorescence image G1 of the mouse A, obtained by acquiring the fluorescence coming from the surface of the mouse A with the image-acquisition unit 15 upon irradiating the mouse A with the excitation light emitted from the illumination device 9.

The display unit 4 is controlled by the control unit 5 to display the fluorescence image G1 stored in the image storage unit 3.

The control unit 5 drives the fluorescence observation apparatus main body 2 and adjusts the exposure time according to the fluorescence level to acquire a fluorescence image G1 of the mouse A in the image storage unit 3. As shown in FIG. 2, the fluorescence image G1 is displayed on the display unit 4. Also, for the fluorescence image G1 displayed on the display unit 4, the control unit 5 can specify a region C, where light is to be blocked, via a specifying unit (not shown) inside the control unit 5.

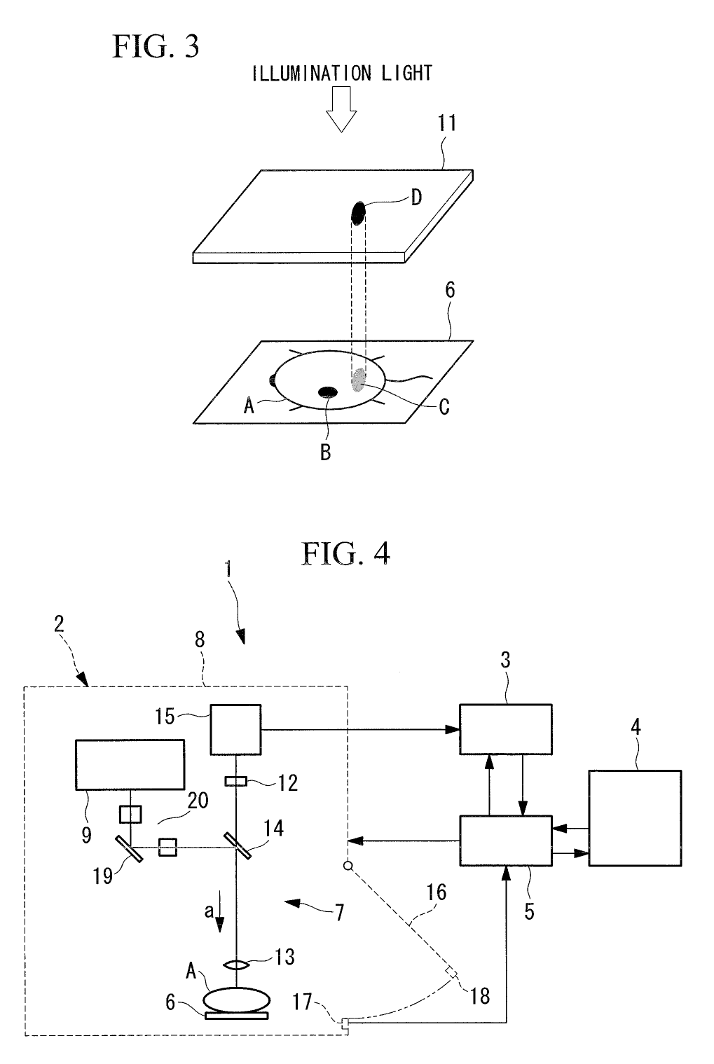

As shown in FIG. 3, the control unit 5 can drive the light-blocking unit 11 to block light for a light-blocking position D corresponding to the specified light-blocking region C.

The light-blocking unit 11 is a liquid-crystal filter (hereinafter referred to as liquid-crystal filter 11) that can control transmission and blocking of light by using a liquid crystal material. The light-blocking unit 11 can drive the liquid crystal at region D corresponding to the position specified in the region C where light is to be blocked, via the control unit 5, to block the light there.

The image storage unit 3, the display unit 4, and the control unit 5 may be devices such as ordinary personal computers.

The operation of the fluorescence observation apparatus 1 according to this embodiment, configured as above, will be described in the following.

To perform fluoroscopy of the mouse A using the fluorescence observation apparatus 1 according to this embodiment, the observer secures the anesthetized mouse A, which has been administered a fluorescent contrast agent, on the stage 6 inside the case 8 of the fluorescence observation apparatus main body 2 with securing means such as tubes or the like, and closes the door 16 in the case 8.

Because the door 16 of the case 8 is provided with the sensor 17, a signal indicating that the door 16 is closed is sent from the sensor 17 to the control unit 5. With the door closed, the case 8 is shielded from external light, thus forming a black box, and therefore, the fluorescence can be more clearly detected.

The control unit 5 sends an activation signal to the fluorescence observation apparatus main body 2 and the image storage unit 3, whereupon acquisition of the fluorescence image G1 is performed by the fluorescence observation apparatus main body 2.

In other words, when excitation light is emitted from the illumination device 9 in the fluorescence observation apparatus main body 2 in response to the activation signal from the control unit 5, it is reflected at the dichroic mirror 14 and is radiated on the mouse A on the stage 6 via the relay optical system 10, the liquid crystal filter 11, and the objective lens 13. At this time, the liquid-crystal filter 11 is turned off, and so all of the excitation light and the reflected light from the mouse A is transmitted.

By radiating excitation light, the fluorescent contrast agent administered to the mouse A is excited, emitting fluorescence, and the emitted fluorescence is collected by the objective lens 13 and is transmitted through the liquid-crystal filter 11, the relay optical system 10, and the dichroic mirror 14, and the fluorescence passing through the image-acquisition optical system 12 is acquired by the image-acquisition unit 15 to obtain the fluorescence image G1. The fluorescence image G1 obtained by the image-acquisition unit 15 is stored in the image storage unit 3 and is displayed on the display unit 4.

The fluorescence image G1 has a lesion B where fluorescence is emitted by the fluorescent contrast agent, which accumulates in tumor tissue, and a site C which although is not a site of interest such as the bladder or liver, is a region where the fluorescent contrast agent accumulates to a high degree, emitting fluorescence.

In other words, the display unit 4 displays both the site of interest B and the region C which emits unwanted fluorescence and where light other than the site of interest should be blocked from the fluorescence image G1.

The observer can ascertain the position of an internal organ in the small laboratory animal based on his or her knowledge and experience and can distinguish between the site of interest B, such as tumor tissue, and the region C.

Therefore, when the observer specifies the discriminated region C using the specifying unit (not shown) while looking at the fluorescence image G1 on the display unit 4, the control unit 5 calculates the position, area, etc. of the region D on the liquid-crystal filter 11 shown in FIG. 3 and controls the liquid-crystal filter 11 to turn the region D on.

The specified light-blocking region D is sent to the fluorescence observation apparatus main body 2 from the control unit 5, and the liquid-crystal filter 11 is driven. Pixels at the position corresponding to the light-blocking region D on the liquid-crystal filter 11 are turned on so as not to transmit light.

Then, when excitation light is emitted from the illumination device 9 in the fluoroscope apparatus main body 2 with the region D on the liquid-crystal filter 11 turned on, it is reflected at the dichroic mirror 14 and passes through the relay optical system 10, and the excitation light transmitted through the portion other than the light-blocking region D on the liquid-crystal filter 11 is radiated onto the mouse A on the stage 6 via the objective lens 13. At this time, the excitation light is not radiated at the portion corresponding to region C which is not the site of interest in the mouse A.

The fluorescence from the mouse A is collected by the objective lens 13, is transmitted through the liquid-crystal filter 11, the relay optical system 10, and the dichroic mirror 14, and passes through the image-acquisition optical system 12, and a fluorescence image G2 in which light in the region C is blocked is acquired by the image-acquisition unit 15. At image acquisition time, a suitable exposure time is set for the site of interest B to obtain the fluorescence image G2.

The fluorescence image G2 obtained by the image-acquisition unit 15 is stored in the image storage unit 3 and is displayed on the display unit 4. Fluorescence for the region C, which is not the site of interest in the obtained fluorescence image G2, is blocked, and therefore, only fluorescence for the site of interest B is present.

With the fluorescence observation apparatus 1 according to this embodiment, by blocking light so that unnecessary strong fluorescence is not generated, for example, from the bladder or kidney, saturation does not occur during image acquisition, and the fluorescence required to be observed does not become obscured, thus enabling acquisition of a fluorescence image.

By setting a suitable exposure time for the area to be observed during image acquisition, microvessels and the like are not overlooked, making it possible to acquire a clear fluorescence image.

In some cases, substances exhibiting autofluorescence are used in the ingredients contained in feed for raising small laboratory animals, and when consuming this feed, it accumulates in the intestinal tract. Then, when observing the small laboratory animal with the fluorescence observation apparatus 1, strong fluorescence is detected from the intestinal tract. In such cases, it is also possible to observe only a site of interest with a similar method.

Furthermore, it is possible to perform observation in the same way also with other substances having an autofluorescence component.

In this embodiment, control of the liquid-crystal filter 11 for blocking light is achieved just by switching it on and off; however, an intermediate light-blocking state may be provided by controlling the voltage.

By providing an intermediate state, it is possible to acquire fluorescence from internal organs etc. that are not the site of interest without completely blocking the light, but just attenuating it. With the position of an internal organ that is not the site of interest serving as a reference observation position, by performing image acquisition together with the fluorescence from the site of interest, it is possible to identify the position at observation time in the fluorescence image.

In this embodiment, the light blocking unit 11 is assumed to be a liquid-crystal filter. However it is not limited thereto; instead, it is possible to use any other type of light-blocking unit such as a light-blocking material whose shape can be changed according to the light-blocking region specified with the specifying unit.

In this embodiment, the light-blocking region is specified with an instruction based on an operation performed by the observer. However, in cases where observation is carried out by repeating fluoroscopy multiple times, where it is known in advance that a substantially fixed result (fluorescence intensity) is obtainable, a region with an arbitrarily set fluorescence level or above may be automatically specified by the control unit 5 and the light may be blocked as required. The exposure time may also be set automatically according to the fluorescence intensity of the region to be observed.

Accordingly, an advantage is afforded in that the observation efficiency is increased and the time required for image acquisition is reduced.

Next, a fluorescence observation apparatus 1 according to a second embodiment of the present invention will be described below with reference to FIG. 4.

The feature of the fluorescence observation apparatus 1 according to this embodiment is that a light-reflecting unit 19 is provided instead of the light-blocking unit 11 in the first embodiment.

The basic configuration of the fluorescence observation apparatus 1 is the same as that of the fluorescence observation apparatus 1 according to the first embodiment (FIG. 1). In this embodiment, parts having the same configuration as those in the first embodiment described above are assigned the same reference numerals, and a description thereof is omitted.

The light-reflecting unit 19 is an ordinary known digital micromirror display (DMD) 19 and is disposed at a position substantially conjugate with respect to the observation site on the mouse A. The DMD 19 is an assembly of minute mirrors, which are not shown. By adjusting the angle of each mirror, it is possible to reflect the illumination light radiated from the illumination device 9 to a desired projection position.

As shown in FIG. 4, in response to the activation signal from the control unit 5, excitation light is emitted from the illumination device 9 in the fluorescence observation apparatus main body 2. The excitation light that passes through a relay optical system 20 falls on the DMD 19, and by adjusting the angle of each mirror, the excitation light is reflected. At this time, the mirrors of the DMD 19 are all driven to reflect the excitation light in the entire area. The excitation light reflected at the mirrors is then reflected at the dichroic mirror 14 to be guided in the direction of the optical axis a of the observation optical system 7, and is radiated onto the mouse A on the stage 6 via the objective lens 13.

The fluorescence emitted from the mouse A is collected by the objective lens 13, is transmitted through the dichroic mirror 14, passes through the image-acquisition optical system 12, and is acquired by the image-acquisition unit 15 to obtain the fluorescence image G1. As shown in FIG. 4, the fluorescence image G1 obtained by the image-acquisition unit 15 is stored in the image storage unit 3 and is displayed on the display unit 4. From the displayed fluorescence image G1, it is possible to specify a region D where light is to be blocked, other than the site of interest, such as the bladder. The specified region D is sent from the control unit 5 to the fluorescence observation apparatus main body 2, and the DMD 19 is driven.

Then, in the same manner, excitation light is emitted from the illumination device 9 in the fluorescence observation apparatus main body 2. The excitation light passes through the relay optical system 20, is reflected by the mirrors 19a in the DMD 19 at positions other than the specified light-blocking region D, is then reflected at the dichroic mirror 14, and is radiated onto the mouse A on the stage 6 via the objective lens 13. At this time, excitation light is not radiated at a region C which is not the site of interest in the mouse A.

The fluorescence from the mouse A is collected by the objective lens 13, is transmitted through the dichroic mirror 14, and passes through the image-acquisition optical system 12, and the fluorescence image G2 in which light is blocked at the region C is acquired by the image-acquisition unit 15. At image acquisition time, a suitable exposure time for the site of interest B is set to obtain the fluorescence image G2.

The fluorescence image G2 obtained by the image-acquisition unit 15 is stored in the image storage unit 3 and is displayed on the display unit 4. Because fluorescence for the region C, which is not the site of interest, in the obtained fluorescence image G2 is blocked, only fluorescence for the site of interest B is present.

In addition to the advantages offered by the first embodiment, the fluorescence observation apparatus 1 according to this embodiment, having such a configuration, provides an advantage in that loss of excitation light is small because it uses a DMD.

In this embodiment, the light-reflecting unit is assumed to be the DMD 19. However, it is not limited thereto; any type of light-reflecting member may be employed.

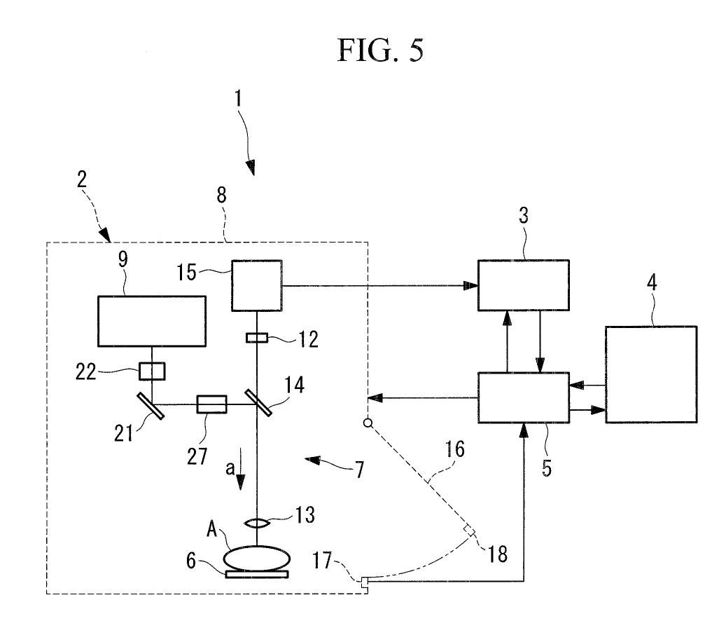

Next, a fluorescence observation apparatus 1 according to a third embodiment of the present invention will be described below with reference to FIG. 5.

The feature of the fluorescence observation apparatus 1 according to this embodiment is provided a light scanning unit 21 and a high-speed shutter 22 instead of the light-reflecting unit in the second embodiment.

The basic configuration of the fluorescence observation apparatus 1 is the same as that in the first embodiment (FIG. 1). In the third embodiment, parts having the same configuration as those in the first embodiment are assigned the same reference numerals, and a description thereof is thus omitted.

The light scanning unit 21 is composed of common known galvanometer mirrors, for example, so-called proximity galvanometer mirrors in which two galvanometer mirrors that each swivel about one axis are placed close to a substantially conjugate plane with respect to a pupil position of the objective lens 13 and are disposed with the two axes orthogonal. The galvanometer mirrors (hereinafter referred to as galvanometer mirrors 21) are swiveled at high speed about the two orthogonal axes based on a control signal from the control unit 5. Accordingly, by oscillating the illumination light incident on the two galvanometer mirrors 21 over respective prescribed angular ranges, the illumination light can be scanned over a region to be observed on the mouse A.

The high-speed shutter 22 is, for example, a common known acousto-optic device. By controlling the open/closed state of the high-speed shutter 22, the illumination light radiated from the illumination device 9 can be radiated only at an arbitrary position of the light scanning unit 21.

An illumination optical system 27 is an optical system for radiating the illumination light from the light scanning unit 21 onto the mouse A via the dichroic mirror 14 and the objective lens 13.

The fluorescence observation apparatus 1 according to this embodiment is, for example, a laser scanning microscope 1. The illumination device 9 is, for example, a laser light source device using a laser diode. The image-acquisition unit 15 is, for example, an optical detector that uses a photomultiplier tube using a high-sensitivity CCD camera.

In response to an activation signal from the control unit 5, excitation light is emitted from the illumination device 9 in the fluorescence observation apparatus main body 2, passes through the high-speed shutter 22, and is incident on the light scanning unit 21. The galvanometer mirrors 21 are swiveled to deflect the incident excitation light within a prescribed angular range. At this time, the high-speed shutter 22 is in the open state, and the galvanometer mirrors 21 scan the excitation light in the entire area. Then, the excitation light passes through the illumination optical system 27, is reflected at the dichroic mirror 14, and is radiated onto the mouse A on the stage 6 via the objective lens 13.

The fluorescence emitted from the mouse A is collected by the objective lens 13, is transmitted through the dichroic mirror 14, passes through the image-acquisition optical system 12, and is acquired by the image-acquisition unit 15 to obtain the fluorescence image G1. The fluorescence image G1 obtained by the image-acquisition unit 15 is stored in the image storage unit 3 and is displayed on the display unit 4.

From the displayed fluorescence image G1, it is possible to specify a region D where light is to be blocked, other than the site of interest, such as the bladder. The specified region D is sent from the control unit 5 to the fluorescence observation apparatus main body 2, and the high-speed shutter 22 and the galvanometer mirrors 21 are driven to radiate the excitation light only on the specified region.

Then, in a similar manner, excitation light is emitted from the illumination device 9 in the fluorescence observation apparatus main body 2. The excitation light that passes through the high-speed shutter 22 is reflected by the galvanometer mirrors 21 only at positions other than the specified light-blocking region D, passes through the illumination optical system 27, is reflected at the dichroic mirror 14, and is radiated onto the mouse A on the stage 6 via the objective lens 13. At this time, excitation light is not radiated at the region C which is not the site of interest on the mouse A.

The fluorescence from the mouse A is collected by the objective lens 13, is transmitted through the dichroic mirror 14, and passes through the image-acquisition optical system 12, and a fluorescence image G2 in which light for the region C is blocked is acquired by the image-acquisition unit 15. At image-acquisition time, a suitable exposure time for the site of interest B is set to obtain the fluorescence image G2.

The fluorescence image G2 obtained by the image-acquisition unit 15 is stored in the image storage unit 3 and is displayed on the display unit 4. Because the fluorescence for the region C which is not the site of interest in the obtained fluorescence image G2 is blocked, only the fluorescence for the site of interest B is present.

In addition to the advantages of the first and second embodiments, because the fluorescence observation apparatus 1 according to this embodiment, having such a configuration, uses the galvanometer mirrors 21, it provides an advantage in that it can scan in the Z direction.

In this embodiment, the high-speed shutter 22 is assumed to be an acousto-optic device. However, it is not limited thereto; any other kind of element that can be controlled at high speed may be employed.

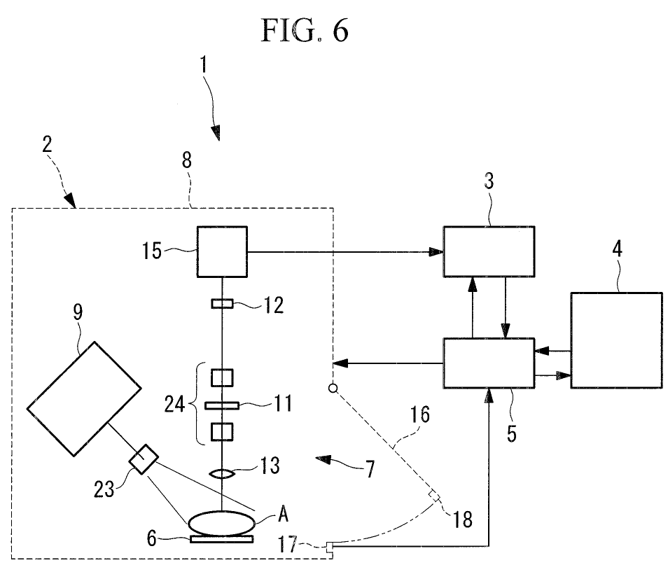

Next, a fluorescence observation apparatus 1 according to a fourth embodiment of the present invention will be described below with reference to FIG. 6.

The feature of the fluorescence observation apparatus 1 according to this embodiment is the positioning of the illumination device and the observation optical system according to the first embodiment are provided in oblique illumination.

The basic configuration of the fluorescence observation apparatus 1 is the same as that of the fluorescence observation apparatus 1 according to the first embodiment (FIG. 1). In the fourth embodiment, parts having the same configuration as those in the first embodiment are assigned the same reference numerals, and a description thereof is omitted here.

The light-blocking unit 11, for example, a liquid-crystal filter 11 identical to that in the first embodiment, can control the light transmission range by switching between on and off states and is disposed at a position substantially conjugate with respect to the observation site on the mouse A.

An illumination optical system 23 is an optical system for irradiating the mouse A with excitation light.

A relay optical system 24, which includes the light-blocking unit 11, is an optical system for introducing the fluorescence from the mouse A, which is collected by the objective lens 13, to the image-acquisition optical system 12.

As shown in FIG. 6, in response to an activation signal from the control unit 5, excitation light is emitted from the illumination device 9 in the fluorescence observation apparatus main body 2 and is introduced to the illumination optical system 23. The introduced excitation light is radiated on the mouse A on the stage 6.

The fluorescence emitted from the mouse A is collected by the objective lens 13 and passes through the relay optical system 24, the liquid-crystal filter 11, and the image-acquisition optical system 12, and the fluorescence is then acquired by the image-acquisition unit 15 to obtain the fluorescence image G1. At this time, the liquid-crystal filter 11 is turned off, and hence, all of the excitation light and the reflected light from the mouse A passes therethrough.

The fluorescence image G1 obtained by the image-acquisition unit 15 is stored in the image storage unit 3 and is displayed on the display unit 4. From the displayed fluorescence image G1, it is possible to specify a region D where the light is to be blocked, other than the site of interest, such as the bladder. The specified region D is sent from the control unit 5 to the fluorescence observation apparatus main body 2, and the liquid-crystal filter 11 is driven. Pixels at positions corresponding to the light-blocking region C on the liquid-crystal filter 11 are turned on so that no light is transmitted therethrough.

Then, in a similar fashion, with the region D on the liquid-crystal filter 11 turned on, the excitation light is emitted from the illumination device 9 in the fluorescence observation apparatus main body 2, passes through the illumination optical system 23, and is radiated on the mouse A on the stage 6.

The fluorescence from the mouse A is collected by the objective lens 13 and passes through the relay optical system 24, excitation light transmitted through portions other than the light-blocking region D on the liquid-crystal filter 11 passes through the image-acquisition optical system 12, and a fluorescence image G2 in which light is blocked at the region C is obtained by the image-acquisition unit 15. At image-acquisition time, a suitable exposure time is set for the site of interest B to obtain the fluorescence image G2.

The fluorescence image G2 obtained by the image-acquisition unit 15 is stored in the image storage unit 3 and is displayed on the display unit 4. Because the fluorescence for the region C, which is not the site of interest, in the obtained fluorescence image G2 is blocked, only fluorescence from the site of interest B is present.

In addition to the advantages of the first to third embodiments, the fluorescence observation apparatus according to this embodiment, having such a configuration, affords an advantage in that it is possible to provide a wide field because it uses oblique illumination.

In this embodiment, the illumination light is radiated from a certain single direction. However, it is not limited thereto; the illumination light may be radiated from any angle and direction.

In this embodiment, the illumination device and the illumination optical system are assumed to form one set. However, it is not limited thereto; a plurality of sets may be provided in order to radiate even brighter illumination light.

In this embodiment, a liquid-crystal filter is used as the light-blocking unit 11 for blocking light, as in the first embodiment. However, the same benefits can also be achieved when employing a system using a light-reflecting unit, like that in the second embodiment, or a light-scanning unit, like that in the third embodiment, in combination with the oblique illumination of this embodiment.

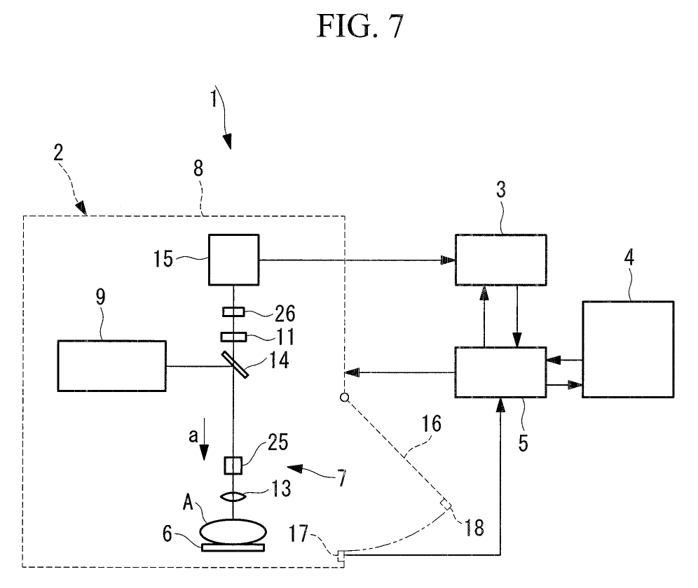

Next, a fluorescence observation apparatus 1 according to a fifth embodiment of the present invention will be described below with reference to FIG. 7.

The feature of the fluorescence observation apparatus according to this embodiment is the positioning of the light-blocking unit 11 of the first embodiment in front of the image-acquisition unit 15, in the light path of the fluorescence instead of in the light path of the illumination light.

The basic configuration of the fluorescence observation apparatus 1 according to this embodiment is the same as that of the fluorescence observation apparatus 1 according to the first embodiment (FIG. 1). In the fifth embodiment, parts having the same configuration as those in the first embodiment are assigned the same reference numerals, and a description thereof shall be omitted here.

The light-blocking unit 11, for example, a liquid-crystal filter 11 identical to that in the first embodiment, can control the transmission range of light by switching between on and off states and is disposed at a position substantially conjugate with respect to the observation site on the mouse A.

A relay optical system 25 is an optical system for introducing the fluorescence from the mouse A, which is collected by the objective lens 13, to the dichroic mirror 14, the liquid-crystal filter 11, and an image-acquisition optical system 26. The image-acquisition optical system 26 is disposed at a position where the fluorescence transmitted through the light-blocking unit 11 is imaged onto the image-acquisition unit 15.

As shown in FIG. 7, in response to an activation signal from the control unit 5, excitation light is emitted from the illumination device 9 in the fluorescence observation apparatus main body 2, is reflected at the dichroic mirror 14 to be guided along the optical axis a of the observation optical system 7, passes through the relay optical system 25, and is radiated on the mouse A on the stage 6 via the objective lens 13.

The fluorescence emitted from the mouse A is collected by the objective lens 13, passes through the relay optical system 25, and is transmitted through the dichroic mirror 14, and the fluorescence passing through the liquid-crystal filter 11 and the image-acquisition optical system 26 is acquired by the image-acquisition unit 15 to obtain the fluorescence image G1. At this time, the liquid-crystal filter 24 is turned off, and hence, all of the fluorescence from the mouse A is transmitted therethrough.

The fluorescence image G1 obtained by the image-acquisition unit 15 is stored in the image storage unit 3 and is displayed on the display unit 4. From the displayed fluorescence image G1, it is possible to specify a region D where light is to be blocked, other than the site of interest, such as the bladder. The specified region D is sent from the control unit 5 to the fluorescence observation apparatus main body 2 to drive the liquid-crystal filter 11. Pixels at positions corresponding to the light-blocking region C on the liquid-crystal filter 11 are turned on, so that no light is transmitted therethrough.

Then, in a similar fashion, the excitation light is emitted from the illumination device 9 in the fluorescence observation apparatus main body 2, is reflected at the dichroic mirror 14, passes through the relay optical system 25, and is radiated onto the mouse A on the stage 6 via the objective lens 13.

The fluorescence from the mouse A is collected by the objective lens 13, passes through the relay optical system 25, and is transmitted through the dichroic mirror 14, the excitation light transmitted through portions other than the light-blocking region D on the liquid-crystal filter 11 passes through the image-acquisition optical system 26, and a fluorescence image G2 in which light is blocked at the region C is obtained by the image-acquisition unit 15. At image acquisition time, a suitable exposure time for the site of interest B is set to obtain the fluorescence image G2.

The fluorescence image G2 obtained by the image-acquisition unit 15 is stored in the image storage unit 3 and is displayed on the display unit 4. Because fluorescence for the region C, which is not the site of interest, in the obtained fluorescence image G2 is blocked, only fluorescence for the site of interest B is present.

In addition to the advantages of the first embodiment, the fluorescence observation apparatus 1 according to this embodiment, having such a configuration, affords the advantage that, with the light-blocking unit 11 disposed in front of the image-acquisition unit 15, the excitation light from the illumination light is not transmitted through the light-blocking unit 11, and therefore, the loss of fluorescence is further reduced.

The above embodiments have been illustrated with the mouse A serving as an example of a small laboratory animal. However, they are not limited thereto; they may be applied to fluoroscopy of any other kind of small laboratory animal, such as a rat or rabbit.

The above embodiments have been described in terms of fluorescence images obtained from small laboratory animals in vivo. However, the present invention is not restricted thereto; for example, it can be suitably applied to ex vivo observation etc. of small laboratory animals.

What is claimed is:

1. A fluorescence observation apparatus comprising:
a light source that emits excitation light;
an optical system that irradiates an image-acquisition site on a small laboratory animal with the excitation light from the light source;
a light-blocking unit that blocks light in a prescribed region of the small laboratory animal or in an image of the prescribed region;
an image-acquisition unit that acquires a fluorescence image of the small laboratory animal; and
a control unit configured to identify a high-fluorescence region having a prescribed fluorescence level or above in the fluorescence image of the small laboratory animal acquired by the image-acquisition unit and to control the light-blocking unit so as to block light at the identified high-fluorescence region.

2. A fluorescence observation apparatus according to claim 1, further comprising:
a display unit that displays the fluorescence image of the small laboratory animal acquired by the image-acquisition unit; and
a specifying unit configured to specify the high- fluorescence region in the fluorescence image displayed by the display unit.

3. A fluorescence observation apparatus according to claim 1, wherein the light-blocking unit is a liquid-crystal filter or a digital micromirror device disposed at a position substantially conjugate with respect to the image-acquisition site on the small laboratory animal.

4. A fluorescence observation apparatus according to claim 1, wherein the light-blocking unit is a galvanometer mirror disposed at a position substantially conjugate with respect to a pupil position of the optical system.

5. A fluoroscopy method comprising:
an irradiating step of irradiating an image-acquisition site on a small laboratory animal with excitation light;
a first image-acquiring step of acquiring a fluorescence image of the small laboratory animal;
an extracting step of extracting a high-fluorescence region with a prescribed fluorescence level or above in the fluorescence image acquired in the first image-acquiring step;
a light-blocking step of blocking light at a high-fluorescence site in the small laboratory animal, corresponding to the high-fluorescence region extracted in the extracting step, or in an image of the high-fluorescence site; and
a second image-acquiring step of acquiring a fluorescence image of the small laboratory animal when the light at the high-fluorescence site of the small laboratory animal, corresponding to the high-fluorescence region, or in the image of the high-fluorescence site is blocked in the light-blocking step.
